(19) United States
(12) Patent Application Publication
(10) Pub. No.: US 2013/0333633 A1
(43) Pub. Date: Dec. 19, 2013

(54) SYSTEMS AND METHODS FOR TESTING DOGS' HEARING, VISION, AND RESPONSIVENESS

(71) Applicant: Tai Cheung Poon, Wanchai (HK)

(72) Inventor: Tai Cheung Poon, Wanchai (HK)

(21) Appl. No.: 13/523,837

(22) Filed: Jun. 14, 2012

(51) Int. Cl. A01K 15/02; A61B 5/12; A61B 3/02; A01K 27/00

(52) U.S. Cl. 119/859; 600/301

(57) ABSTRACT

Systems and methods for testing hearing, vision, and responsiveness of a dog are provided. The systems' components comprise a first, second, third, and fourth projection screen installed on four walls of a confined area; a series of radio-frequency identification (“rfid”) readers installed in a grid pattern on each of the first, the second, the third, and the fourth walls; an rfid tag mounted on a dog collar to be worn by the dog; an accelerometer mounted on the dog collar; a speaker installed on each of the first, second, third, and fourth walls; a processor; and a memory. The system generally uses visual and audio stimuli in order to test the hearing, vision, and physical responsiveness of the dog. The dog's actions are measured by the rfid readers' detecting the movement of the rfid tag on the dog's collar.

### FIELD OF THE INVENTION

This invention is generally related to the training of domestic animals.

More specifically, this invention is related to electronically testing a dog's physical responsiveness with respect to their vision, hearing, and leaping capability.

### BACKGROUND OF THE INVENTION

For most dog owners, their dog is more than just a pet. Unlike other animals, dogs have become domesticated and can live the same home environment as their owners. Dog owners may desire for their dogs to behave in ways that make them pleasant to be around, keep them safe, and provides for the safety of other humans and pets. However, in order to increase obedience and discipline, an owner must spend many hours training the dog. Fundamentally, one should always remember that no human standard should be applied to the dogs. There can be no assumption that the dog will always be disciplined and able to understand exactly what the owner really expects of it.

In dog training, it is important to communicate with the dog in a humane way that the dog comprehends. The underlying principle of all communication is simple: reward desired behavior while ignoring or correcting undesired behavior.

To professional dog trainers, the most effective way to train a dog is for the owner to use and reinforce the techniques taught to the dog repeatedly. Traditionally, owners and dogs may attend class together to learn more about each other and how to work together under a trainer's guidance. Training of such can be effective only if all those who handle the dog take part in the training to ensure consistent commands, methods, and enforcement. Classes may help socialize a dog to other people and dogs. However, training should be more systematic. It is difficult for a dog owner to attend training classes all the time. When dog owners aren't available, the dogs should be capable of being trained by themselves. In view of the foregoing demand, a system and method is desirable on the market, in order to train more systematically and effectively.

In view of the foregoing, there is a need for a system and method that can automatically perform training and testing techniques electronically, without the need for an owner or trainer to be present.

### SUMMARY OF THE INVENTION

According to embodiments of the invention, a dog training system for testing hearing, vision, and responsiveness of a dog is provided. The systems components include: a first projection screen installed on a first wall of a confined area, a second projection screen installed on a second wall of the confined area, a third projection screen installed on a third wall of the confined area, a fourth projection screen installed on a fourth wall of the confined area, a series of radio-frequency identification (“rfid”) readers installed in a grid pattern on each of the first, the second, the third, and the fourth walls, an rfid tag mounted on a dog collar to be worn by the dog, an accelerometer mounted on the dog collar, a speaker installed on each of the first, second, third, and fourth walls, a processor, and a memory.

The four projection screens are synchronized with each other and are configured to simulate environments using images displayed in a panoramic view.

The combined set of the series of sensors on each wall are used to detect precise location of objects that carry rfid tags.

The memory stores instructions that cause the processor to execute a method. The method generally employs the following steps, in no specific or particular order.

The method begins by testing the vision of the dog. The first step of the vision test involves producing panoramic images on the first, second, third, and fourth screens to simulate a certain environment. The second step involves producing a stimulus on the first wall, and measuring how fast the dog moves to the first wall using the series of rfid readers on the first wall.

Next, the hearing of the dog is tested. The hearing test involves emitting a sound from the first speaker, and measuring how fast the dog moves to the first wall. The measurements are taken using the series of rfid readers on the first wall.

Continuing with the method, the physical responsiveness of the dog is tested. The first step in testing physical responsiveness involves producing panoramic images on the first, second, third, and fourth projection screens to simulate a certain environment. The test proceeds with showing a fast moving object moving from a bottom of the first projection screen to a top portion of the first projection screen. Then, the series of rfid readers on the first wall are used for measuring how fast the dog moves from the bottom to the top portion.

In another embodiment of the disclosed invention, a dog training system for testing dogs' capability in hearing, vision, and responsiveness is provided. The systems components are: first, second, third, and fourth projection screens installed on each of the walls of the confined area; a series of rfid readers installed in a grid pattern on each of the first, the second, the third, and the fourth walls; an rfid tag mounted on a dog collar to be worn by the dog; an accelerometer mounted on the dog collar; a speaker installed on each of the first, second, third, and fourth walls; a processor; and a memory.

The four projection screens are synchronized with each other and are configured to simulate environments using images displayed in a panoramic view. The combined set of the series of sensors on each wall are used to detect precise location of objects that carry rfid tags.

The memory stores instructions that cause the processor to execute a method. The method generally employs the following steps, in no specific or particular order.

The method begins by testing the vision of the dog. The first step of the vision test involves producing panoramic images on the first, second, third, and fourth screens to simulate a certain environment. The second step involves producing a stimulus on the first wall, and measuring how fast the dog moves to the first wall using the series of rfid readers on the first wall. Next, a determination is made as to whether the dog has passed or failed the vision test. If, according to the series of rfid readers on the first wall, the dog fails to move to the wall in a pre-specified time, the dog is regarded as having failed the vision test.

Next, the hearing of the dog is tested. The hearing test involves emitting a sound from the first speaker, and measuring how fast the dog moves to the first wall. The measurements are taken using the series of rfid readers on the first wall. Then, a determination is made as to whether the dog has passed or failed the hearing test. If, according to the series of rfid readers on the first wall, the dog fails to move to the wall in a pre-specified time, the dog is regarded as having failed the hearing test.

Continuing with the method, the physical responsiveness of the dog is tested. The first step in testing physical responsiveness involves producing panoramic images on the first, second, third, and fourth projection screens to simulate a certain environment. The test proceeds with showing a fast moving object moving from a bottom of the first projection screen to a top portion of the first projection screen. Then, the series of rfid readers on the first wall are used for measuring how fast the dog moves from the bottom to the top portion. Next, a determination is made as to whether the dog has passed or failed the leaping capability test. If, according to the series of rfid readers on the first wall, the dog fails to leap towards the top portion of the screen in a pre-specified time, the dog is regarded as having failed the leaping test.

In another embodiment of the disclosed invention, a dog training system is provided. The system employs a four-walled confined area, a dog collar, a projection screen on each wall, and a plurality of rfid readers disposed on each wall. The dog collar is to be worn by a dog, and may have an rfid tag and an accelerometer. The projection screens are configured to display certain stimuli in order elicit a physical response from the dog. The rfid readers are configured such that three-dimensional movement of the rfid tag can be measured upon the physical response of the dog. Further, the orientation of the dog may also be tracked and measured. Orientation measurements are carried out by the accelerometer.

In a further embodiment, the system may further comprise a speaker disposed on each of the four walls wherein the speakers are configured to emit sounds in order to elicit a physical response from the dog.

The system may employ a memory for storing configuration data and measured data regarding testing. The system may also employ a processor for processing the configuration data and the measured data stored on the memory. The functioning of the system may be carried out using the processor and memory.

It is, therefore, an objective of the disclosed invention to provide a system and method for testing dogs' vision, hearing, and responsiveness.

In accordance with these and other objects which will become apparent hereinafter, the invention will now be described with particular reference to the drawing.

### BRIEF DESCRIPTION OF THE DRAWINGS

FIG. 1 shows a schematic view of a confined area of a dog training system according to an embodiment of the present invention.

### DETAILED DESCRIPTION

Referring now to the figure, a schematic diagram of an exemplary overview of a dog training system is shown. The diagram shown is a mere example of one of many possible arrangements of the systems and methods of the disclosed invention.

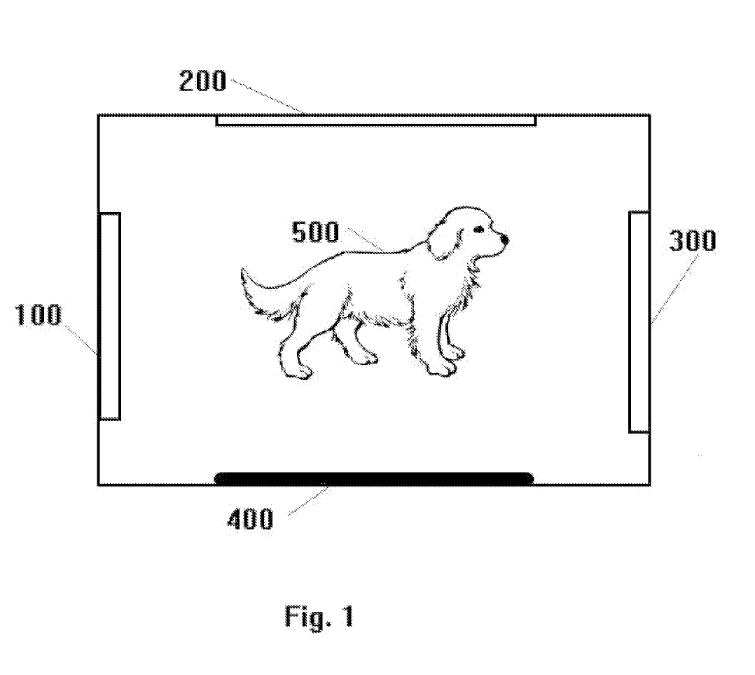

Referring still to FIG. 1, a confined area of a dog training system according to an embodiment of the present invention is shown. The systems employs a first projection screen 100 installed on a first wall of the confined area, a second projection screen 200 installed on a second wall of the confined area, a third projection screen 300 installed on a third wall of the confined area, a fourth projection screen 400 installed on a fourth wall of the confined area. The system further comprises a series of radio-frequency identification (“rfid”) readers installed in a grid pattern on each of the first, the second, the third, and the fourth walls, an rfid tag mounted on a dog collar to be worn by a dog 500, an accelerometer mounted on the dog collar, a speaker installed on each of the first, second, third, and fourth walls, a processor; and a memory.

The four projection screens are synchronized with each other and are configured to simulate environments using images displayed in a panoramic view. The combined set of the series of sensors on each wall are used to detect precise location of objects that carry rfid tags.

The memory stores instructions that cause the processor to execute a method. The method generally employs the following steps, in no specific or particular order.

The method begins by testing the vision of the dog 500. The first step of the vision test involves producing panoramic images on the first, second, third, and fourth screens to simulate a certain environment. The second step involves producing a stimulus on the first wall, and measuring how fast the dog 500 moves to the first wall using the series of rfid readers on the first wall.

Next, the hearing of the dog 500 is tested. The hearing test involves emitting a sound from the first speaker, and measuring how fast the dog 500 moves to the first wall. The measurements are taken using the series of rfid readers on the first wall.

Continuing with the method, the physical responsiveness of the dog 500 is tested. The first step in testing physical responsiveness involves producing panoramic images on the first, second, third, and fourth projection screens to simulate a certain environment. The test proceeds with showing a fast moving object moving from a bottom of the first projection screen to a top portion of the first projection screen. Then, the series of rfid readers on the first wall are used for measuring how fast the dog 500 moves from the bottom to the top portion.

In another embodiment of the disclosed invention, a dog 500 training system for testing dogs' capability in hearing, vision, and responsiveness is provided. The systems components are: first, second, third, and fourth projection screens installed on each of the walls of the confined area; a series of rfid readers installed in a grid pattern on each of the first, second, third, and fourth walls; an rfid tag mounted on a dog collar to be worn by the dog 500; an accelerometer mounted on the dog collar; a speaker installed on each of the first, second, third, and fourth walls; a processor; and a memory.

The four projection screens are synchronized with each other and are configured to simulate environments using images displayed in a panoramic view. The combined set of the series of sensors on each wall are used to detect precise location of objects that carry rfid tags.

The memory stores instructions that cause the processor to execute a method. The method generally employs the following steps, in no specific or particular order.

The method begins by testing the vision of the dog 500. The first step of the vision test involves producing panoramic images on the first, second, third, and fourth screens to simulate a certain environment. The second step involves producing a stimulus on the first wall, and measuring how fast the dog 500 moves to the first wall using the series of rfid readers on the first wall. Next, a determination is made as to whether the dog 500 has passed or failed the vision test. If, according to the series of rfid readers on the first wall, the dog 500 fails to move to the wall in a pre-specified time, the dog is regarded as having failed the vision test.

Next, the hearing of the dog 500 is tested. The hearing test involves emitting a sound from the first speaker, and measuring how fast the dog moves to the first wall. The measurements are taken using the series of rfid readers on the first wall. Then, a determination is made as to whether the dog 500 has passed or failed the hearing test. If, according to the series of rfid readers on the first wall, the dog 500 fails to move to the wall in a pre-specified time, the dog is regarded as having failed the hearing test.

Continuing with the method, the physical responsiveness of the dog 500 is tested. The first step in testing physical responsiveness involves producing panoramic images on the first, second, third, and fourth projection screens to simulate a certain environment. The test proceeds with showing a fast moving object moving from a bottom of the first projection screen to a top portion of the first projection screen. Then, the series of rfid readers on the first wall are used for measuring how fast the dog 500 moves from the bottom to the top portion. Next, a determination is made as to whether the dog 500 has passed or failed the leaping capability test. If, according to the series of rfid readers on the first wall, the dog 500 fails to leap towards the top portion of the screen in a pre-specified time, the dog is regarded as having failed the leaping test.

In another embodiment of the disclosed invention, a dog training system is provided. The system employs a four-walled confined area, a dog collar, a projection screen on each wall, and a plurality of rfid readers disposed on each wall. The dog collar is to be worn by a dog 500, and may have an rfid tag and an accelerometer. The projection screens are configured to display certain stimuli in order elicit a physical response from the dog 500. The rfid readers are configured such that three-dimensional movement of the rfid tag can be measured upon the physical response of the dog 500. Further, the orientation of the dog 500 may also be tracked and measured. Orientation measurements are carried out by the accelerometer.

In a further embodiment, the system may further comprise a speaker disposed on each of the four walls wherein the speakers are configured to emit sounds in order to elicit a physical response from the dog 500.

The system may employ a memory for storing configuration data and measured data regarding testing. The system may also employ a processor for processing the configuration data and the measured data stored on the memory. The functioning of the system may be carried out using the processor and memory.

Radio frequency identification (“rfid”) tagging is a known method of identification. An information carrying device, or tag, functions in response to a coded radio frequency (“rf”) signal transmitted from a base station or reader. The rf carrier signal reflects from the tag and can be demodulated to recover information stored in the tag. The tag typically includes a semiconductor chip having rf circuits, logic, and memory, as well as an antenna. Various tag structures, circuits, and programming protocols are known in the art.

Most rf systems typically have three components: (1) a tag or tag (the item being identified), (2) an interrogator or reader, and (3) a data managing medium (typically including cabling, computers, and software which tie together the tags and interrogators into a useful solution). Most rf products are typically designed to detect tags when they pass within a predefined range of the reader.

There are generally two types of rf tags known in the art: passive rf tags and active rf tags. Passive rf tags, unlike active ones, do not require a battery in order to transmit a rf signal frequency. Instead, passive rf tags rely on an external source to provoke signal transmission. The rf reader transmits the operating power for these tags. As a result, such passive rf systems generally have a detection range of limited to a couple meters. However, passive rf tags may generally be manufactured to be smaller in size than active rf tags due to the absence of a battery.

Most passive rfid systems work as follows. A reader emits an electromagnetic field for the purpose of powering the tag. A coil in the tag is powered by the electromagnetic field, causing the tag's circuitry to “wake up.” The tag uses this power to send an identifying signal back to the interrogator.

While the disclosed invention has been taught with specific reference to the above embodiments, a person having ordinary skill in the art will recognize that changes can be made in form and detail without departing from the spirit and the scope of the invention. The described embodiments are to be considered in all respects only as illustrative and not restrictive. All changes that come within the meaning and range of equivalency of the claims are to be embraced within their scope. Combinations of any of the methods, systems, and devices described hereinabove are also contemplated and within the scope of the invention.

What is claimed is:

1. A dog training system for testing hearing, vision, and responsiveness of a dog, comprising:
a first projection screen installed on a first wall of a confined area;
a second projection screen installed on a second wall of the confined area;
a third projection screen installed on a third wall of the confined area;
a fourth projection screen installed on a fourth wall of the confined area, wherein the fourth projection screen is synchronized with the first, second, and third projection screens to simulate an environment using images displayed in a panoramic format;
a series of rfid readers installed in a grid pattern on each of the first, the second, the third, and the fourth walls, wherein all of the rfid readers are used to detect a precise location of rfid tags;
an rfid tag mounted on a dog collar to be worn by the dog;
an accelerometer mounted on the dog collar, wherein the accelerometer detects orientation of the dog;
a speaker installed on each of the first, second, third, and fourth walls;
a processor; and
a memory storing instructions that cause the processor to execute a method, the method comprising:
testing vision of the dog, wherein the vision testing comprises;
producing panoramic images on the first, second, third, and fourth screens to simulate a certain environment; and
producing a stimulus on the first wall, and measuring how fast the dog moves to the first wall using the series of rfid readers on the first wall;
testing hearing of the dog, wherein the hearing testing comprises:
emitting a sound from the first speaker, and measuring how fast the dog moves to the first wall using the series of rfid readers on the first wall;
testing physical responsiveness of the dog, wherein the physical responsiveness testing comprises:
producing panoramic images on the first, second, third, and fourth projection screens to simulate a certain environment; and
showing a fast moving object moving from a bottom portion of the first projection screen to a top portion of the first projection screen, and measuring how fast the dog moves from the bottom portion to the top portion using the series of rfid readers on the first wall.

2. A dog training system for testing dogs' capability in hearing, vision, and responsiveness, comprising:
a first projection screen installed on a first wall of a confined area;
a second projection screen installed on a second wall of the confined area;
a third projection screen installed on a third wall of the confined area;
a fourth projection screen installed on a fourth wall of the confined area, wherein the fourth projection screen is synchronized with the first, second, and third projection screens to simulate environments using images displayed in panoramic format;
a series of rfid readers installed in a grid pattern on each of the first, second, third, and fourth walls, wherein the series of rfid readers can be used to detect a precise location of objects that carry rfid tags;
an rfid tag mounted on a dog collar to be worn by a dog in the confined area;
an accelerometer mounted on the dog collar, wherein the accelerometer detects orientation of the dog;
a speaker installed on each of the first, second, third, and fourth walls;
a processor; and
a memory storing instructions that cause the processor to execute a method, the method comprising:
testing vision of the dog, wherein the vision testing comprises;
producing panoramic images on the first, second, third, and fourth screens to simulate a certain environment;
producing a stimulus on one of the walls, and measuring how fast the dog moves to the that wall using all of the series of rfid readers; and
if, according to the series of rfid readers on the first wall, the dog fails to move to the wall in a pre-specified time, the dog is regarded as having failed the vision testing;
testing hearing of the dog, wherein the hearing testing comprises:
emitting a sound from one of the speakers, and measuring how fast the dog moves to that speaker using all of the series of rfid readers; and
if, according to the rfid readers, the dog fails to move to the speaker in a pre-specified time, the dog is regarded as having failed the hearing test;
testing leaping capability of the dog, wherein the leaping capability testing comprises:
producing panoramic images on the first, second, third, and fourth projection screens to simulate a certain environment;
showing a fast moving object moving from a bottom portion of one of the projection screens to a top portion of that projection screen, and measuring how fast the dog moves from the bottom portion to the top portion using all of the series of rfid readers;
if, according to the rfid readers, the dog fails to leap towards the top portion of the screen in a pre-specified time, the dog is regarded as having failed the leaping capability test; and
repeating the leaping capability test to measure any improvements.

3. A dog training system, comprising:
a confined area with four walls;
a dog collar to be worn by a dog wherein the dog collar comprises an rfid tag;
a projection screen disposed on each of the four walls wherein each projection screen is configured to display certain stimuli in order elicit a physical response from the dog; and
a plurality of rfid readers disposed on each wall wherein the rfid readers are configured such that movement of the rfid tag is measured.

4. The dog training system of claim 3, further comprising a speaker disposed on each of the four walls wherein the speakers are configured to emit sounds in order to elicit a physical response from the dog.

5. The dog training system of claim 3, wherein orientation of the dog is tracked using an accelerometer disposed on the dog collar.

6. The dog training system of claim 4, wherein orientation of the dog is tracked using an accelerometer disposed on the dog collar.

7. The system of claim 3, further comprising a memory for storing configuration data and measured data regarding testing; and a processor for processing the configuration data and the measured data stored on the memory.
